(19) United States
(12) Patent Application Publication
(10) Pub. No.: US 2009/0234311 A1
(43) Pub. Date: Sep. 17, 2009

(54) Urine collection device

(76) Inventor: Judith Holloway, New Hope, PA (US)

Correspondence Address: JAMES RAY & ASSOCIATES, 2640 PITCAIRN ROAD, MONROEVILLE, PA, 15148, US

(21) Appl. No.: 12/454,924

(22) Filed: May 26, 2009

Related U.S. Application Data
(63) Continuation-in-part of application No. 12/283,445, filed on Sep. 12, 2008.
(60) Provisional application No. 60/971,592, filed on Sep. 12, 2007.

(51) Int. Cl. A61F 5/44

(52) U.S. Cl. 604/328

(57) ABSTRACT

A lighted stent urine collection device is used to collect urine that would otherwise leak along the stent. The collection device includes an opening to allow placement of the stents, an inverted open channel to position them, and means to secure the stents, allowing for otherwise leaked urine to collect in the bag.

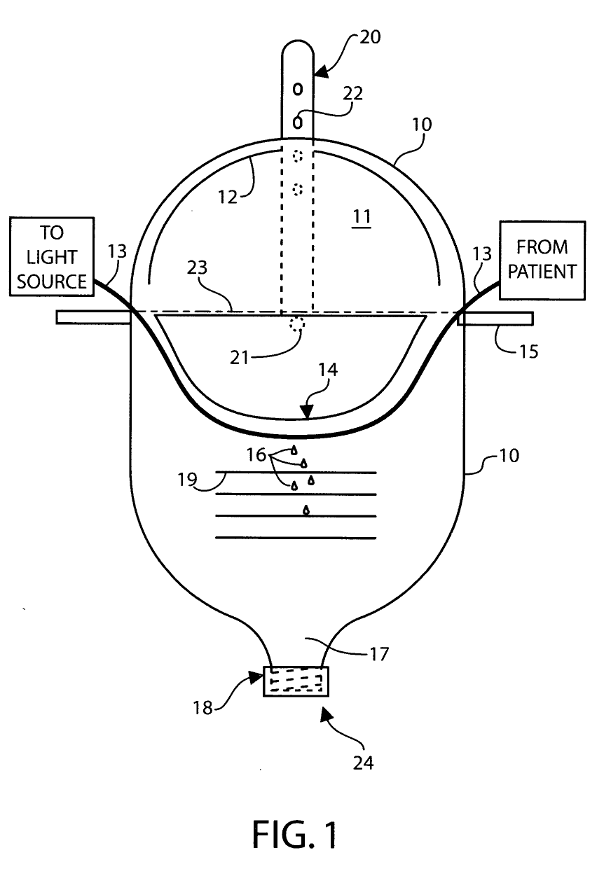

### CROSS REFERENCE TO RELATED APPLICATION

This patent application is related to and claims priority from U.S. Provisional Patent Application Ser. No. 60/971,592 filed Sep. 12, 2007 and is a Continuation-In-Part of U.S. patent application Ser. No. 12/283,445 filed Sep. 12, 2008.

### FIELD OF THE INVENTION

The present invention relates, in general, to urine collection and, more particularly, this invention relates to a urine collection device for use with a catheter when a lighted stent is used.

### BACKGROUND OF THE INVENTION

Prior to the conception and development of the present invention, as is generally well known in the prior art from a Foley catheter system, a ureteral stent/catheter is typically inserted, laparoscopically or otherwise, through the urethra and into the ureters. When a fiberoptic element is introduced into that catheter and connected to an infrared light source, urine leaks outside of the catheter system. The Foley catheter is a tube inserted into the bladder to continuously drain urine. Typically the Foley catheter drains the urine from the patient's bladder but urine leaks along the exterior stent which is connected to a light source.

The problem of urine leakage has been addressed by U.S. Pat. No. 4,248,214 issued to Hannah et al. which provides for a drainage adapter. However, this adapter is for a urethral catheter to illuminate the bladder. A ureteral catheter which is finer and longer is inserted up into the kidney(s) and usually drains into the bladder. However, when a lighted stent is introduced into the ureteral catheter and connected to a light source, the urine leaks.

U.S. Pat. No. 5,531,741 issued to Barbacci teaches the lighted stent but does not allow for urine collection with the device.

### SUMMARY OF THE INVENTION

The present invention provides for a collection device for urine that otherwise would leak from the stent used in laparoscopic surgery or diagnostic procedures. The instant invention is a urine collection device for a lighted stent with ureteral catheter. The urine collection device includes a fluid-impervious latex-free collection bag formed from a first predetermined material. There is an open flap top having a first predetermined size and shape. Further, there is a means to close such flap top. An inverted arc shaped ridge/channel having a second predetermined size and shape is provided to accommodate at least one such lighted stent. Also, there is a stent retention means disposed generally adjacent each side of the top opening.

### OBJECTS OF THE INVENTION

It is, therefore, one of the primary objects of the present invention to provide a urine collection device used with lighted stents.

Another object of the present invention is to provide a device that collects urine leaking in laparoscopic surgery and diagnostic procedures.

Still another object of the present invention is to provide a device that will allow for accurate urine output measurement.

Yet another object of the present invention is to provide a device that optionally collects leaked urine and is in fluid communication with a further drainage tube.

An important object of the present invention is that it maintains the integrity of the closed Foley drainage system by providing an alternate collection for the stent urine.

An additional object of the present invention is to provide a collection bag that is secured to the patient's leg, to prevent free movement.

In addition to the various objects and advantages of the present invention described with some degree of specificity above, it should be obvious that additional objects and advantages of the present invention will become more readily apparent to those persons who are skilled in the relevant art from the following more detailed description of the invention, particularly when such description is taken in conjunction with the attached drawing figures and with the appended claims.

### BRIEF DESCRIPTION OF THE DRAWINGS

The single FIGURE is a side elevation view of the device.

### BRIEF DESCRIPTION OF A PRESENTLY PREFERRED AND VARIOUS ALTERNATIVE EMBODIMENTS OF THE INVENTION

Reference is now made, more particularly, to the FIGURE. Illustrated therein is a urine collection bag 10. The collection bag 10 is preferably manufactured from a clear, fluid-impervious, latex-free material. Most preferably it is produced from plastic. The collection bag 10 includes an open flap 11 at the top thereof which can be closed with an adhesive strip 12 or other single fastener. One or two lighted stents 13 such as those used in laparoscopic surgery can be inserted at the fold line 23 and fitted under the inverted arc ridge/channel 14 in the interior of the bag 10. In this position the urine running along the stent 13 drops into the bag 10, as shown by number 16.

The stent(s) 13 is further secured by tapes 15 on the lateral sides of the bag 10. Preferably one side of these would have an inwardly facing surface coated with a pressure sensitive adhesive.

Preferably the collection bag 10 has an aperture 17 through the bottom of the bag 10 and a means 24 for closing the aperture 17, such as a cap 18 or a conventional Foley drainage tubing system (not shown). When used with the Foley tubing, the collection bag 10 would be in fluid communication with a system separate from the Foley catheter.

The collection bag 10 preferably has lines as measurement 19 printed thereon. Whether the collection bag 10 is used with a cap 18 or connected to a Foley bag system (not shown), so that an accurate measurement of urine output can be made.

A securing means is attached to the collection bag 10 at a predetermined location to secure it against free movement. Preferably an elastic strap 20 is attached at the back of the bag. The strap preferably has a button and hole closure 21 with pre-made series of holes 22 for fitting on the patient's leg.

It can be seen from the above description that the present invention captures the urine by providing a collection bag which allows the stent(s) to pass through and along an interior ridge/channel in the shape of an inverted arc. This allows the urine to drop by gravity into the bag. The top is a flap which can be sealed over, with small flaps on the sides of the bag to also hold the stent(s) in place. Preferably these would have an adhesive strip, but snaps or hook and loop type fasteners could also be used.

The collection bag has an aperture in the bottom of the bag with a fitting that accommodates a cap or a Foley collection tubing so that the collection device, tubing, and Foley bag are in fluid communication. This is separate from the actual Foley catheter and collection system that may also be in place for surgery.

The bag may have measurement marks printed on the bag corresponding to the volume of the bag. When used with a cap, the patient's urine volume output can be accurately measured since the leaked urine is captured and measured. If the case is long the bag can be connected to a regular Foley collection bag system and measured in that bag. This output is separate from and in addition to urine collected from the bladder by the Foley catheter.

The bag has a securing means. Preferably the securing device is a strap attached to the back of the bag which can secure it to the patient's inner thigh, to prevent free movement or swinging, and keep it out of the operative field.

While a presently preferred and various alternative embodiments of the present invention have been described in sufficient detail above to enable a person in the relevant art to make and use the same it should be obvious that various other adaptations and modifications can be envisioned by those persons skilled in such art without departing from either the spirit of the invention or the scope of the appended claims.

I claim:

1. A urine collection device for having a lighted stent with a ureteral catheter, said device comprising:
(a) a fluid-impervious latex-free collection bag formed from a first predetermined material;
(b) an open flap top having a first predetermined size and shape;
(c) means engageable with at least a portion of said flap and at least a portion of said collection bag for closing said flap;
(d) an inverted arc shaped ridge/channel of having a second predetermined size and shape to accommodate at least one such lighted stent; and
(e) a stent retention means disposed generally adjacent each side of top opening for retaining said at least one lighted stent in place.

2. A urine collection device, according to claim 1, wherein said collection bag has lines printed on said collection bag as measurement marks corresponding to a volume of urine collected.

3. A urine collection, according to claim 1, wherein said stent retention means are a flap top and tapes attached to said collection bag wherein said top and tapes have an inwardly facing side coated with a pressure sensitive adhesive.

4. A urine collection, according to claim 3, wherein said stent retention means has a pair of said tapes at each side of said collection bag.

5. A urine collection, according to claim 1, wherein said arched ridge/channel accommodates two stents.

6. A urine collection, according to claim 1, wherein said collection bag has an aperture at a predetermined location in a bottom of said collection bag, further having a fitting, said fitting has a predetermined shape and size to receive one of a cap and drainage tubing.

7. A urine collection device of claim 1 further having collection bag securing means attached to a back of said collection bag to secure said collection bag from free movement.

8. A urine collection, according to claim 1, wherein said collection bag retention means is a strap having a predetermined length.

9. A urine collection, according to claim 8, wherein said strap is formed from a stretchable material with and has a series of holes and one of a hook and button for fastening.
